(19) United States
(12) Patent Application Publication
(10) Pub. No.: US 2023/0002801 A1
(43) Pub. Date: Jan. 5, 2023

(54) OFFLINE AND INLINE DETERMINATION OF CONCENTRATION OF METABOLITES IN CELL CULTURE FLUID

(71) Applicant: Repligen Corporation, Waltham, MA (US)

(72) Inventor: Amit Kumar Dutta, Waltham, MA (US)

(73) Assignee: Repligen Corporation, Waltham, MA (US)

(21) Appl. No.: 17/573,336

(22) Filed: Jan. 11, 2022

Related U.S. Application Data
(60) Provisional application No. 63/136,477, filed on Jan. 12, 2021.

(51) Int. Cl. C12Q 1/06; G01N 23/10; G01N 21/05

(52) U.S. Cl. CPC C12Q 1/06; G01N 23/10; G01N 21/05; G01N 2021/1748

(57) ABSTRACT

Devices, systems, and methods described herein relate to determining a concentration of a species of interest in a sample by using a spectrometer. For example, a concentration of a species of interest may be determined by passing a first feed of a sample with a species of interest through a flow-through variable pathlength spectrophotometer and reading a first absorbance value. A change in the concentration of the species of interest may be effected in the sample, and a second feed of the sample may be passed through a flow through variable pathlength spectrophotometer. A second absorbance value may be read. The difference between the first absorbance value and the second absorbance value may be used to determine the concentration of the species of interest.

### CROSS-REFERENCE TO RELATED APPLICATIONS

This is a nonprovisional of provisional application Ser. No. 63/136,477, filed Jan. 12, 2021, the entirety of which application is incorporated by reference herein.

### FIELD

The present disclosure pertains to the use of spectroscopy, specifically a flow cell spectrometer.

### BACKGROUND

Spectroscopic analysis is a broad field in which the composition and properties of a material in any phase, gas, liquid, solid, are determined from the electromagnetic spectra arising from the interaction (e.g., absorption, luminescence, or emission) with energy. One aspect of spectrochemical analysis, known as spectroscopy, involves interaction of radiant energy with the material of interest. The particular methods used to study such matter-radiation interactions define many sub-fields of spectroscopy. One field in particular is known as absorption spectroscopy, in which the optical absorption spectra of liquid substances are measured. The absorption spectra is the distribution of light attenuation (due to absorbance) as a function of light wavelength. In a simple spectrophotometer the sample substance which is to be studied is placed in a transparent container, also known as a cuvette or sample cell. Electromagnetic radiation (light) of a known wavelength, λ, (i.e. ultraviolet, infrared, visible, etc.) and intensity I is incident on one side of the cuvette. A detector, which measures the intensity of the exiting light, I, is placed on the opposite side of the cuvette. The length that the light propagates through the sample is the distance d. Most standard UV/visible spectrophotometers utilize standard cuvettes which have 1 cm path lengths and normally hold 50 to 2000 μL of sample. For a sample consisting of a single homogeneous substance with a concentration c, the light transmitted through the sample will follow a relationship know as Beer's Law: A=εcl where A is the absorbance (also known as the optical density (OD) of the sample at wavelength λ where OD=the −log of the ratio of transmitted light to the incident light), ε is the absorptivity or extinction coefficient (normally at constant at a given wavelength), c is the concentration of the sample and “l” is the path length of light through the sample.

Spectroscopic measurements of samples are widely used in various fields. Often the species of interest in a sample is highly concentrated. For example, certain biological samples, such as proteins, DNA or RNA are often isolated in concentrations that fall outside the linear range of the spectrophotometer when absorbance is measured. Therefore, dilution of the sample is often required to measure an absorbance value that falls within the linear range of the instrument. Frequently multiple dilutions of the sample are required which leads to both dilution errors and the removal of the sample diluted for any downstream application. It is, therefore, desirable to take existing samples with no knowledge of the possible concentration and measure the absorption of these samples without dilution.

Multiple sample cuvettes may solve the problem of repetitive sampling. However, this approach still requires the preparation of multiple sample cuvettes and removes some samples from further use. Furthermore, in most spectrophotometers the path length, “l”, is fixed.

Another approach to the dilution problem is to reduce the path length in making the absorbance measurement. By reducing the measurement path length, the sample volume can be reduced. Reduction of the path length also decreases the measured absorption proportionally to the path length decrease. For example, a reduction of path length from the standard 1 cm to a path length of 0.2 mm provides a virtual fifty-fold dilution. Therefore, the absorbance of more highly concentrated samples can be measured within the linear range of the instrument if the path length of the light travelling through the sample is decreased. There are several companies that manufacture cuvettes that while maintaining the 1 cm or 1 cm² dimension of standard cuvettes decrease the path length through the sample by decreasing the interior volume. By decreasing the interior volume, less sample is required and a more concentrated sample can be measured within the linear range of most standard spectrophotometers. While these low volume cuvettes enable the measurement of more concentrated samples, the path length within these cuvettes is still fixed. Thus, if the sample concentration falls outside the linear range of the spectrophotometer the sample still may need to be diluted or another cuvette with an even smaller path length may be required before an accurate absorbance reading can be made.

### SUMMARY

The foregoing listing is intended to be exemplary rather than exhaustive, and those of skill in the art will appreciate that other aspects and embodiments not described above are within the scope of the present disclosure.

The present disclosure, in its various aspects, provides methods, systems, and devices for determining the concentration of cell metabolites in cell culture fluid. Knowledge of the cell metabolite concentration is important in order to maintain optimum growth conditions. Current methods often must be performed offline and require sample dilution. The present disclosure describes, as an example, a method of determining cell metabolite concentrations inline and without the need to dilute the sample.

In an aspect, embodiments of the disclosure describe a method of determining a sample concentration. This method may comprise passing a first feed through a flow-through variable pathlength spectrophotometer, wherein the feed comprises a sample and impurities. The method may comprise reading a first absorbance value and passing a second feed through the flow-through variable pathlength spectrophotometer, wherein the feed comprises the sample. The methods may comprise reading a second absorbance value, wherein the difference between the first absorbance value and the second absorbance value comprises the sample concentration.

In various embodiments described herein and otherwise within the scope of the disclosure, the non-treated feed may comprise a cell culture fluid. Reading the first and second absorbance values may comprise measuring the absorbance at 280 nm. The affinity column may comprise a Protein A affinity column. The affinity column may comprise a lactate dehydrogenase (LDH) affinity column. The affinity column may be configured to operate for at least 500 cycles.

In an aspect, embodiments of the disclosure describe a method for determining a sample concentration. The method may comprise passing a non-treated feed through a variable pathlength spectrophotometer, wherein the non-treated feed comprises a sample and impurities. The method may comprise reading a first absorbance value and passing the non-treated feed through an affinity column, wherein the resulting fluid comprises a treated feed. The method may comprise passing the treated feed through the variable pathlength spectrophotometer and reading a second absorbance value, where the difference between the first absorbance value and the second absorbance value comprises the sample concentration.

In an aspect, embodiments of the disclosure describe a method for determining a sample concentration. The method may comprise passing a first fluid through a flow cell spectrometer, where the first fluid comprises a sample and impurities, reading a first absorbance value, passing the first fluid through an affinity column, resulting in a second fluid, passing the second fluid through the flow cell spectrometer, reading a second absorbance value, and measuring a third absorbance value proportional to the difference between the first absorbance value and the second absorbance value.

In an aspect, embodiments of the disclosure describe a method for determining a sample concentration. The method may comprise passing a non-treated feed fluid through a flow cell spectrometer, wherein the first fluid comprises a sample and impurities, reading a first absorbance value, passing the non-treated feed fluid through an affinity column, resulting in a treated feed fluid, passing the treated feed fluid through the flow cell spectrometer, reading a second absorbance value, and measuring a third absorbance value proportional to the difference between the first absorbance value and the second absorbance value.

In an aspect, embodiments of the disclosure describe a method of determining a sample concentration. The method may comprise passing a non-treated feed through a variable pathlength spectrophotometer, wherein the non-treated feed comprises a sample and impurities. The method may comprise reading a first absorbance value and mixing the non-treated feed with a reagent, wherein mixing further comprises causing a reaction which produces a product. The method may comprise passing the product through the variable pathlength spectrophotometer and reading a second absorbance value, wherein the difference between the first absorbance value and the second absorbance value is proportional to the sample concentration.

In an aspect, embodiments of the disclosure describe a method of determining a sample concentration. The method may comprise passing a first fluid through a flow cell spectrometer, wherein the first fluid comprises a sample and impurities. The method may comprise reading a first absorbance value and mixing the first fluid with a second fluid, wherein mixing further comprises causing a reaction which produces a product. The method may comprise passing the product through the flow cell spectrometer, reading a second absorbance value, and measuring a third absorbance value proportional to the difference between the first absorbance value and the second absorbance value.

In an aspect, embodiments of the disclosure describe a method of determining a sample concentration. The method may comprise passing a non-treated feed fluid through a flow cell spectrometer, wherein the non-treated feed fluid comprises a sample and impurities and reading a first absorbance value. The method may comprise mixing the non-treated fluid feed with a reagent, wherein mixing further comprises causing a reaction which produces a product, passing the treated feed fluid through the flow cell spectrometer, and reading a second absorbance value. The method may comprise measuring a third absorbance value proportional to the difference between the first absorbance value and the second absorbance value.

In various embodiments described herein and otherwise within the scope of the disclosure, the non-treated feed may comprise a cell culture fluid. The reagent may comprise glucose oxidase, peroxidase, 4-aminopherazone, and phenol. Reading the first and second absorbance values may comprise measuring the absorbance at 505 nm. The reagent may comprise reduced nicotinamide adenine dinucleotide (NADH). Reading the first and second absorbance values may comprise measuring the absorbance at 340 nm. The reagent may comprise L-lactate oxidase, 4-amino antipyrine, peroxidase, and N-ethyl-N-(2-hydroxy-3-sulfopropyl)-3-methylaniline (TOOS). The product may comprise quinoneimine dye. Reading the first and second absorbance values may comprise measuring the absorbance at 550 nm.

In some embodiments, a method of determining a sample concentration includes passing a first feed through a flow-through variable pathlength spectrophotometer, wherein the first feed comprises a sample and impurities, reading a first absorbance value, passing a second feed of the sample through the flow-through variable pathlength spectrophotometer, wherein the second feed comprises the impurities, and reading a second absorbance value. The difference between the first absorbance value and the second absorbance value can comprise a concentration of the sample.

The method may further include determining a third absorbance value corresponding to a difference between the first absorbance value and the second absorbance value and using the third absorbance value to determine the concentration of the sample.

The first feed can be a cell culture fluid. Reading the first and second absorbance values can include measuring the respective absorbances at 280 nm. The method may further include passing the first feed through an affinity column prior to passing the first feed through the flow-through variable pathlength spectrophotometer. In some embodiments the affinity column is a lactate dehydrogenase (LDH) affinity column or a Protein A affinity column.

In some embodiments, a method of determining a sample concentration may include passing a first fluid through a flow cell spectrometer, where the non-treated feed comprises a sample and impurities, reading a first absorbance value, and passing the non-treated feed through an affinity column, wherein the resulting fluid comprises a treated feed. The method may further include passing the treated fluid through the flow cell spectrometer, and reading a second absorbance value, where the difference between the first absorbance value and the second absorbance value comprises a concentration of the sample.

The method may further include determining a third absorbance value corresponding to a difference between the first absorbance value and the second absorbance value and using the third absorbance value to determine the concentration of the sample. In some embodiments the first feed is a cell culture fluid.

In some embodiments, reading the first and second absorbance values comprises measuring the respective absorbances at 280 nm. In some embodiments, the affinity column is a lactate dehydrogenase (LDH) affinity column or a Protein A affinity column. In some embodiments, the flow cell spectrometer is a flow through variable pathlength spectrophotometer.

In some embodiments, a method of determining a sample concentration includes passing a first fluid through a flow cell spectrometer, wherein the first fluid comprises a sample and impurities, reading a first absorbance value, mixing the first fluid with a second fluid, wherein mixing further comprises causing a reaction which produces a product, passing the product through the flow cell spectrometer, and reading a second absorbance value, where the difference between the first absorbance value and the second absorbance value is proportional to a concentration of the sample.

In some embodiments the first fluid is a non-treated feed fluid. In some embodiments the second fluid includes a reagent. In some embodiments, the reagent includes glucose oxidase, peroxidase, 4-aminopherazone, and phenol. In some embodiments, the reagent includes reduced nicotinamide adenine dinucleotide (NADH). In some embodiments, the reagent incudes L-lactate oxidase, 4-amino antipyrine, peroxidase, and N-ethyl-N-(2-hydroxy-3-sulfopropyl)-3-methylaniline (TOOS).

The method can further include determining a third absorbance value corresponding to a difference between the first absorbance value and the second absorbance value and using the third absorbance value to determine the concentration of the sample. In some embodiments, reading the first and second absorbance values comprises measuring the absorbance at 340 nm, 505 nm, or 550 nm. In some embodiments, the product includes quinoneimine dye.

### DESCRIPTIONS OF FIGURES

FIG. 1 depicts a system used to determine concentration of a cell metabolite using treated and non-treated fluid feed.

FIG. 2 depicts a system used to determine concentration of a cell metabolite using non-treated fluid feed and a reagent.

FIG. 3 depicts a system used to determine concentration of product using non-treated fluid feed and feed passed through an affinity column.

FIG. 4 depicts a system used to determine concentration of glucose.

FIG. 5 depicts a system used to determine concentration of lactate dehydrogenase (LDH) using a mixer.

FIG. 6 depicts a system used to determine concentration of LDH using an affinity column.

FIG. 7 depicts a system used to determine concentration of lactate.

### DETAILED DESCRIPTION

The present disclosure provides, among other things, systems and methods that enable determination of determination of a concentration of a species of interest in sample without sample dilution. In general, methods may use a variable path length spectrophotometer to determine concentrations of cell metabolites in a sample. Furthermore, certain methods of the present disclosure do not require that the path length be known to determine the concentration of samples.

Methods disclosed herein may operate offline and/or in-line, enabling estimation of concentration of species of interest with increased efficiency and/or risk of error in measurements.

In various methods, multiple sample feeds, each with a different treatment of a species of interest, may be sequentially processed by a variable path length spectrophotometer, for example, in a continuous through flow. Differences in measured absorbances across time between the feeds may be used to estimate respective differences in concentrations of a species of interest between the feeds.

Some methods disclosed herein may analyze a single feed comprising an unknown concentration of a species of interest via use of a variable path length spectrophotometer (for example, but not limited to, C-Tech Solo VPE or C-Tech Flow VPE, manufactured by Repligen, Bridgewater, N.J. 08807), wherein at least one reagent is introduced to the feed along a time range within the period of measurement. Differences in measured absorbance of the feed over time may be used to estimate the concentration of the species of interest based on the effect of the reagent.

Accordingly, methods of the presentation may enable estimation of an unknown concentration of a species of interest in a sample. This and other objects and advantages of the disclosure, as well as additional inventive features, will be apparent from the description of the disclosure provided herein.

The term “moving the probe relative to the vessel” or “moving the probe relative to the sample” means that the vessel or the sample relative to the probe is moved. This encompasses situations where the probe is moving and the vessel or sample is stationary, the vessel or sample is moving and the probe is stationary, and where the sample or the vessel is moving, and the probe is moving.

The term “taking an absorbance reading” means that any absorbance reading(s) is measured by the device or instrument. This encompasses situations where the absorbance reading is taken at a single wavelength and/or a single path length or where the reading is taken at multiple wavelengths (such as in a scan) and/or multiple path lengths.

The term “sample(s)” may include, but is not limited to, compounds, mixtures, surfaces, solutions, emulsions, suspensions, cell cultures, fermentation cultures, cells, tissues, secretions, and extracts. In many examples, a sample may be a fluid sample.

The term “feed” will be understood to encompass a sample, and in many embodiments, a flowing sample.

The term “non-treated feed” may be a feed containing a species of interest which has not been subject to a treatment to effect a change in the concentration of the species of interest.

The term “species” may include, but is not limited to, a compound, a cell metabolite, or other molecule of interest. A species of interest may be organic or inorganic.

The term “motor” is any device that can be controlled to provide a variable path length through a sample.

Some embodiments of this disclosure relate to estimation of concentration of a species of interest in a sample. For example, a concentration of a cell metabolite such as an antibody, lactate dehydrogenase (LDH), or lactate in a cell culture may be related to the performance of the cell culture. In another example, a concentration of glucose in cell culture fluid may reflect a growth condition of the cell culture fluid. Monitoring the concentration of a species of interest may thus be valuable for practitioners to monitor performance of a sample and/or to determine procedures necessary to maintain optimum conditions.

However, determination of a concentration of a species of interest at a discrete point in time may be difficult due to the presence of impurities and/or other molecules in the sample, which may contribute to background noise.

Furthermore, standard spectrophotometric analysis requires an offline step, wherein a subsample of a sample is removed from a process and analyzed independently. However, removal of the sample for analysis presents risk of contamination, requires significant time and effort on the part of a practitioner, and is limited in its ability to generate real-time estimations of a concentration of a species during a process. If the sample is highly concentrated such that measured absorbance values fall outside the linear range of a spectrophotometric analysis, the sample may need to be diluted or a cuvette with a smaller path length found. Each of these steps may present further risks of contamination and delays to analysis.

Methods and systems described herein may allow for estimation of a concentration of a species of interest without sample dilution, without removal of impurities, and/or without interference to a process flow. Without wishing to be bound by any theory, methods and systems may pertain to offline and/or inline determination of species concentration with respect to a feed.

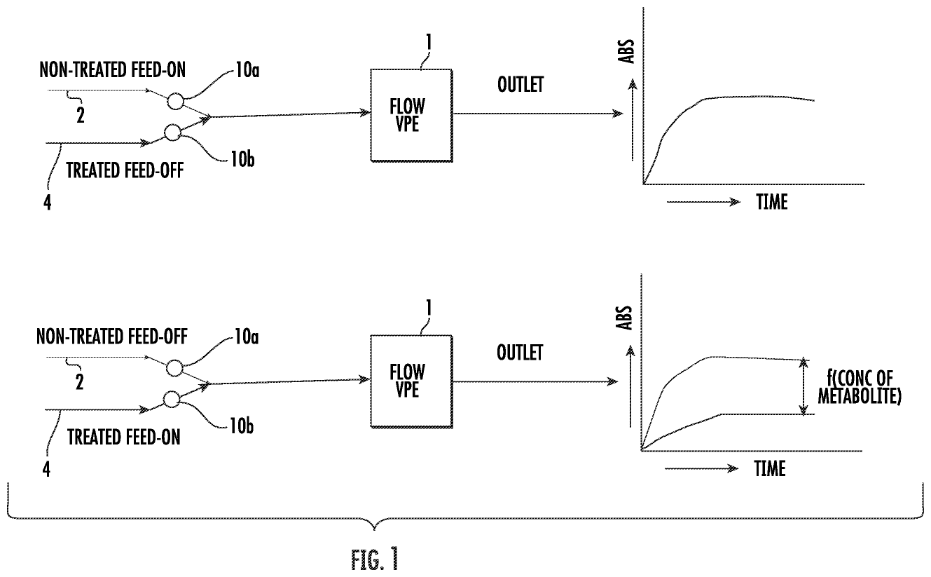

For example, a flow-through variable path length spectrophotometer 1 may be used to estimate a concentration of a species of interest by measuring differences in absorbances between non-treated and treated samples 2, 4, as illustrated in FIG. 1. A non-treated feed or sample 2 may comprise, in many embodiments, a cell culture fluid. A treatment may be, in various embodiments, installed in fluid communication with a feed and with a flow-through variable path length spectrophotometer 1. The flow-through variable path length spectrophotometer 1 may be installed in fluid communication with prior and/or subsequent processing of a feed. Accordingly, absorbance measurements of the effect of the treatment may be an inline measurement, not requiring removal of a sample from a process flow. Alternatively, absorbance measurements may be performed offline.

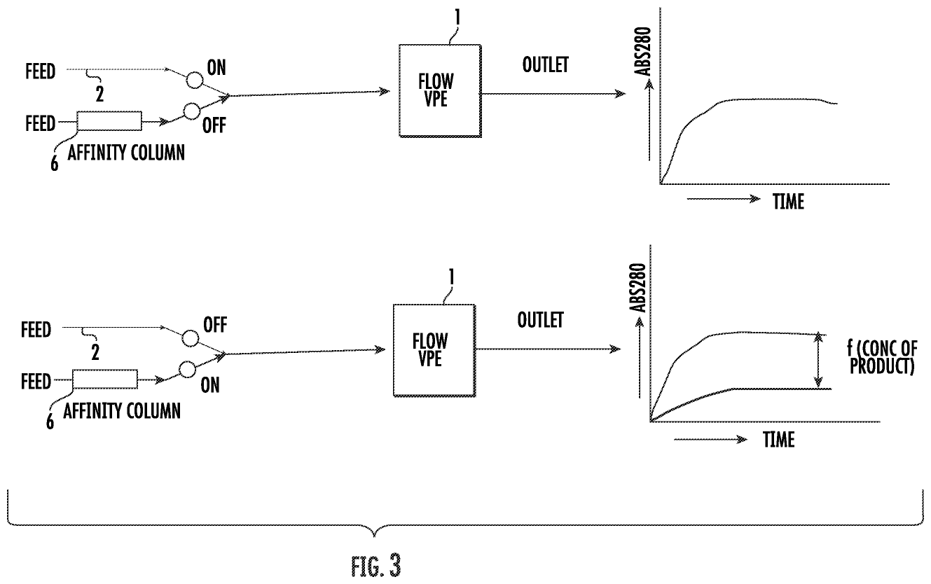

In various embodiments, a treatment may comprise adding or depleting a species of interest from a sample, for example, via a filtration system or chromatography column. In some embodiments, a treatment may include an affinity column 6, as illustrated in FIG. 3. An affinity column 6 may be configured to deplete a species of interest from a feed. Accordingly, the treated sample may comprise the filtrate. For example, a species of interest may be Protein A. In various examples, an affinity column 6 may be a Protein A affinity column configured to deplete Protein A from a feed. In many embodiments, an affinity column 6 may be configured to operate for at least 500 cycles, and/or until it loses 60% of its capacity. In some embodiments, an affinity column 6 may be configured to operate for at least 300-700 cycles, or any iterative number of cycles in between. In some embodiments, an affinity column 6 may be configured to operate until it loses 40-80% of its capacity, or any iterative percentage in between. In examples wherein a species of interest is LDH, a treatment may be an LDH affinity column, for example, comprising ε-aminohexanoyl-NAD+.

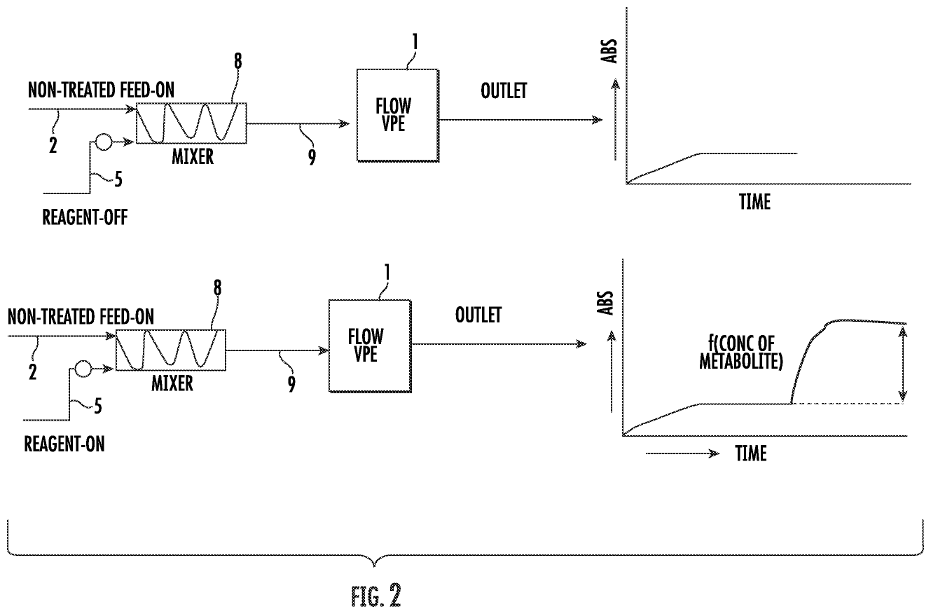

Alternatively, or additionally, a treatment may comprise a mixer 8 as illustrated in FIG. 2, which may be used, for example, to mix a feed of interest with one or more reagents, additional feeds, or additional species. At least one reagent, additional feed, or additional species may effect a change in the concentration of the species of interest. In many embodiments, at least one reagent, additional feed, or additional species may react with the species of interest so as to generate a product with a distinguishable absorbance within a linear range corresponding to standards of a spectrophotometer. For example, a reaction may result in a dye with a known absorbance, such as quinoneimine dye, which may be observable at 550 nm.

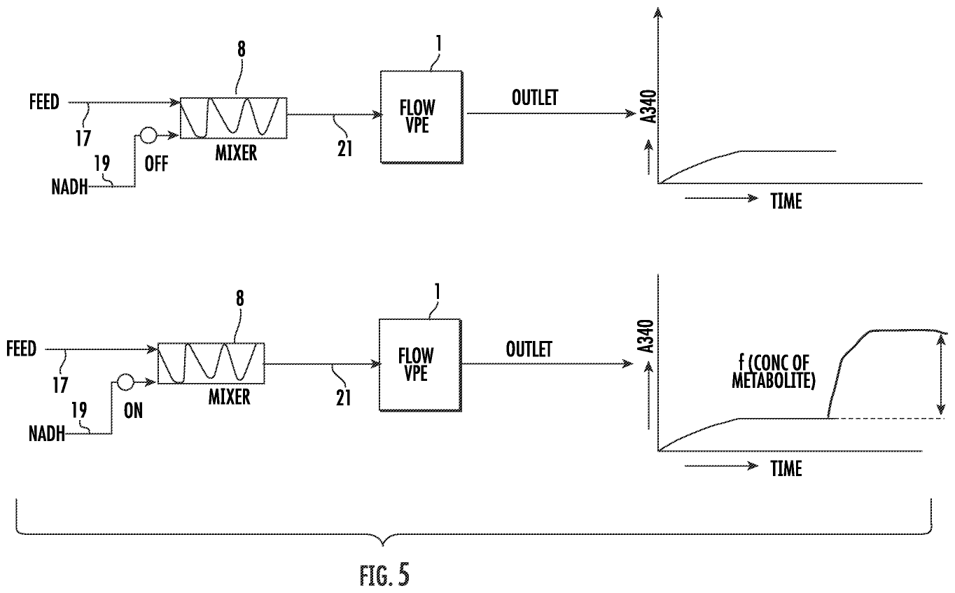

In one non-limiting example embodiment, the variable pathlength spectrophotometer 1 and associated software of U.S. Pat. No. 9,046,485 to Salerno et al. (hereinafter “Salerno '485”), incorporated by reference in its entirety herein, may be used. For example, the flow-through system illustrated in FIG. 5 and described in column 10, lines 39-65 of Salerno '485 may be used in conjunction with methods presently disclosed as flow cell spectrometer.

It will be understood that one or more pumps (not shown) may be used to control flow in and/or out of any of the inlet or outlet of the feed lines described herein. For example, a flowrate, flux, pressure, viscosity, or the like of a fluid at a feed line may be adjusted by controlling one or more pumps by a frequency, speed, force, stroke length, pressure adjustment, or the like. It will be understood that flow through any of the feed lines described herein may be adjusted manually or automatically, for example, via a controller (not shown).

Spectrophotometer settings and/or standards, such as a wavelength and/or pathlength for a reading, may be determined prior to an absorbance reading of the contents of a flow cell. In many examples, standards may be determined according to slope spectroscopy standard determination methods described in U.S. Pat. No. 10,830,778 of Salerno et al. (hereinafter “Salerno '778”), incorporated by reference in its entirety herein. For example, absorbance readings may be recorded while moving a probe relative to a sample. Settings of wavelength and pathlength may be set for a feed of interest offline and/or prior to commencement of a treatment of the feed.

Alternatively, or additionally, a wavelength may be determined based upon a known industry standard or characteristic of a desired reaction product. For example, a feed containing glucose may be analyzed at a wavelength of 505 nm based on (a) an ability of 4-(p-benzochinone-monoimino)-phenazone to be registered at a wavelength of 505 nm, and (b) on an intent to generate said 4-(p-benzochinone-monoimino)-phenazone using the glucose-containing sample using reagents comprising glucose oxidase, peroxidase, 4-aminophenazone, and phenol. In non-limiting examples, fluid feeds as described herein may be analyzed at 280 nm, 340 nm, 505 nm, or 550 nm.

Based on determined standards for a feed corresponding to a species of interest, a wavelength and pathlength may be set for a flow cell spectrometer, or a flow-through variable pathlength spectrophotometer, fluidly connected to a feed line comprising a non-treated sample. Absorbance may be measured for the feed over time.

Subsequently, absorbance may be measured for a treated feed. In some embodiments, a non-treated feed may be redirected upstream of the spectrophotometer to a treatment, which may be either rejoined or separately joined in fluid communication with the flow cell of the spectrophotometer.

For example, in an offline mode as illustrated in FIG. 1, a feed line containing a species of interest may split into a first path 2 without a filtration component and a second path 4 with a treatment. The split feed line may be configured to allow flow through only a single path at a time, for example, through alternative direction by one or more valves 10a, 10b or switches. The first and second paths 2, 4 may each be coupled to a flow cell of a flow-through variable length spectrophotometer 1. According to various embodiments described herein, a split feed line may first be configured to pass fluid flow of a sample through the first path, and absorbance readings may be taken thereof.

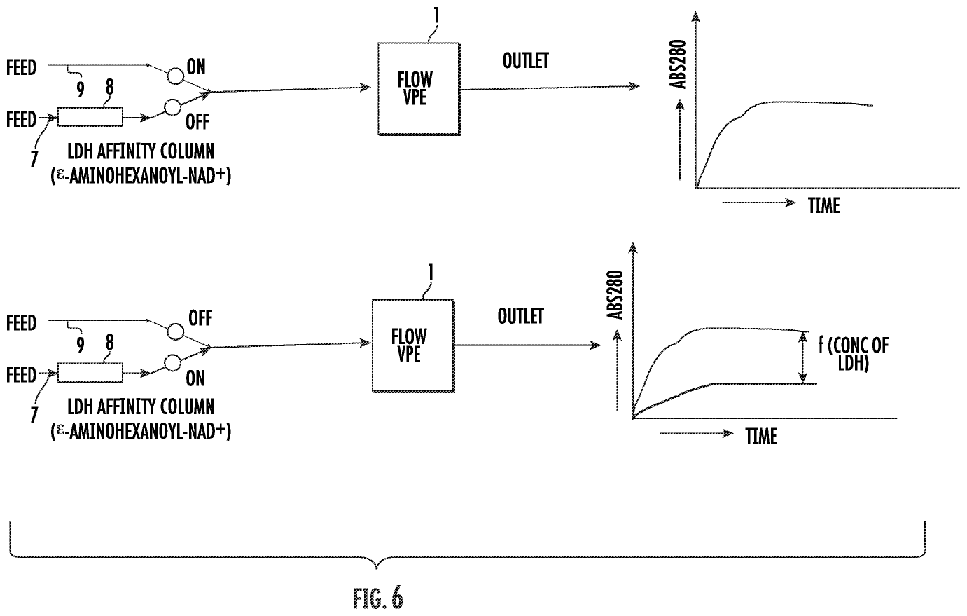

Subsequently, for example, upon an occurrence of a predicted behavior of the absorbance readings over time or at a predetermined time point, flow may be redirected from the first path 2 to the second path 4 comprising the treatment. In many embodiments, a treatment may comprise a chromatography column. For example, a predicted behavior of the absorbance readings may comprise a stabilization or plateau of the readings. The treatment may affect the concentration of a species of interest within the feed. For example, the filtration component may be an affinity column 6 configured to substantially remove the species of interest from the feed, as illustrated in FIGS. 3 and 6.

The filtrate may pass through the flow cell of the flow-through variable length spectrophotometer 1, and absorbance readings may be observed thereof. Based on a respective occurrence of a predicted behavior of the absorbance readings over time or at a respective predetermined time point, as described above, absorbance readings may be compared between the respective feed flows through the first and second paths 2, 4. In some embodiments, a fit line or predictive model may be applied to the absorbance readings of the feed through the first path, and an absorbance reading of the feed through the second path may be compared to a predicted point of the fit line or predictive model.

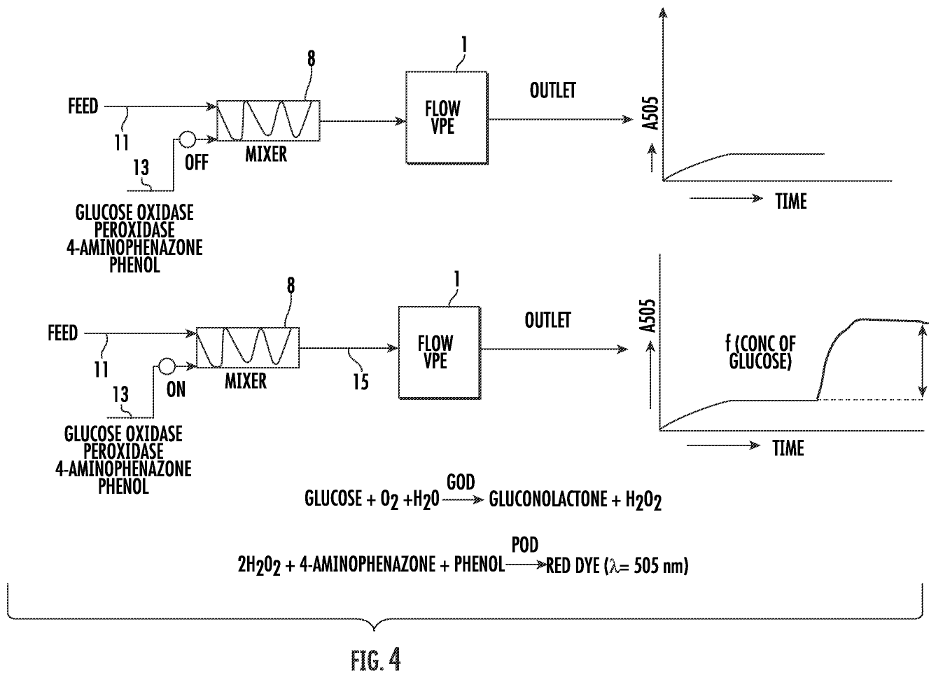

In an exemplary inline mode as illustrated in FIG. 2, a first outlet comprising a feed of a species of interest and a second outlet 5 comprising at least one reagent may feed into a mixer or a mixing chamber 8. FIGS. 4, 5, and 7 further comprise systems and methods with mixing chambers 8 as described herein. The first and second outlets may be independently operable and unidirectional, for example, to prevent backflow of a reagent through the first outlet. The mixing chamber 8 may facilitate interaction of the feed of the species of interest 2 with the at least one reagent 5. In some embodiments, the mixing chamber 8 may comprise one or more mechanisms suitable for effecting and/or expediting a reaction between the feed of the species of interest 2 with the at least one reagent 5. For example, the mixing chamber 8 may comprise, in various embodiments, a stirring mechanism. In another example, a mixing chamber may comprise a heating element.

A single outlet 9 may proceed from the mixing chamber 8 and be fluidly coupled with the flow cell of a flow-through variable length spectrophotometer 1.

A sample 2 may be passed through the first outlet, through the mixing chamber 8, and through the flow cell of the flow-through variable length spectrophotometer 1 while the second outlet (with reagent 5) remains switched off. Absorbance readings may be observed for the sample over time. Subsequently, for example, upon an occurrence of a predicted behavior of the absorbance readings over time or at a predetermined time point, flow of at least one reagent 5 may be begun through the second outlet into the mixing chamber 8.

The feed of the species of interest 2 and the at least one reagent 5 may be mixed in the mixing chamber 8. The combination thereof, including any products of reactions between the species of interest and the at least one reagent, may pass through the flow cell of the flow-through variable length spectrophotometer 1, and absorbance readings may be observed thereof.

Based on a respective occurrence of a predicted behavior of the absorbance readings over time or at a respective predetermined time point, as described above, absorbance readings may be compared between the respective feed flows comprising only the feed of the species of interest and the combination of the feed of the species of interest with the reagent. In other words, a first absorbance value corresponding to a non-treated feed may be compared to a second absorbance value corresponding to a treated feed. A third absorbance value corresponding to the difference between the first and second absorbance values may be used to determine the concentration of a species of interest in the non-treated feed.

The measurement and comparison of multiple absorbance values between treated and non-treated feeds may reduce concern over the quantitative accuracy of single readings due to the presences of impurities, as each treated and non-treated feed may be expected to contain similar or the same impurities.

After collection of measurements for determination of concentration of a species of interest in a feed, feeds may be redirected to an original process flow without interruption of the feeds. For example, in the exemplary offline model described above, feed may be switched from the second path with the filtration component to the first path without the filtration component. In the exemplary inline model described above, reagent flow through the second outlet may be stopped.

It will be understood that systems described herein may comprise one or more additional feeds or components coupled to the flow cell of a flow-through variable pathlength spectrophotometer or to an inlet thereof, which may, for example, be useful for washing or cleaning the flow cell or an inlet thereof. Accordingly, risk of contamination by a species of interest and resulting misleading absorbance readings may be decreased during readings for a feed in which the concentration of the species of interest has been depleted.

While many examples described herein are described with respect to downstream processing steps, it will be readily understood that methods and/or systems may be implemented in upstream processes. For example, upstream validation of a concentration of a species of interest may be useful in quality control measures. Furthermore, exemplary processes may further include predicating steps to determine slope spectroscopy standards relevant for a feed of interest, for example, in accordance with Salerno '778.

Furthermore, it will be understood that devices and systems useful for implementing the methods discussed herein are presently contemplated. For example, various feed lines, valves, ports, inlets, outlets, mixing chambers, stirring mechanisms (e.g., magnetic stirring mechanisms, mechanical stirring mechanisms, or other effective stirring mechanism), automated and/or manual controllers, sensors, or other system(s) or device(s) useful for fluidly connecting and/or otherwise implementing methods described herein are presently contemplated. Each may be individually, or in any combination, sterilizable and/or packageable. Various components may be single-use, disposable, and/or multi-use. Embodiments are not limited herein.

### EXAMPLES

Absorbance of a feed containing an unknown concentration of LDH is read at 280 nm, as illustrated in FIG. 6. The feed 7 may be directed through an LDH affinity column 6, which may contain ε-aminohexanoyl-NAD+. The absorbance of the filtrate may be read at 280 nm. The concentration of LDH in the initial feed 9 is determined by the difference in the absorbance signals at 280 nm.

Absorbance of a first feed 9 containing an unknown concentration of LDH is read at 280 nm. A second feed 7 containing the same sample of the first feed may be directed in parallel through an LDH affinity column 6, which may contain ε-aminohexanoyl-NAD+. The absorbance of the filtrate of the second feed 7 may be read at 280 nm. The concentration of LDH in the initial feed 9 is determined by the difference in the absorbance signals at 280 nm.

Absorbance of a first feed 11 containing glucose is read at 505 nm, as illustrated in FIG. 4. Glucose 11 is mixed via mixer 8 with reagents 13 including glucose oxidase, peroxidase, 4-aminophenazone, and phenol. The resulting product 15 contains 4-(p-benzochinone-monoimino)-phenazone, which can be estimated by reading the absorbance at 505 nm. The concentration of glucose in the first feed 11 is determined by the difference in absorbance signals at 505 nm.

Absorbance of a first feed 17 containing an unknown concentration of LDH is read at 340 nm, as illustrated in FIG. 5. The first feed 17 is mixed via mixer 8 with a second feed 19 containing reduced nicotinamide adenine dinucleotide (NADH). The resulting product 21 contains NAD+. The absorbance of the product 21 can be read at 340 nm. The initial unknown concentration of LDH is determined by the difference in absorbance signals at 340 nm.

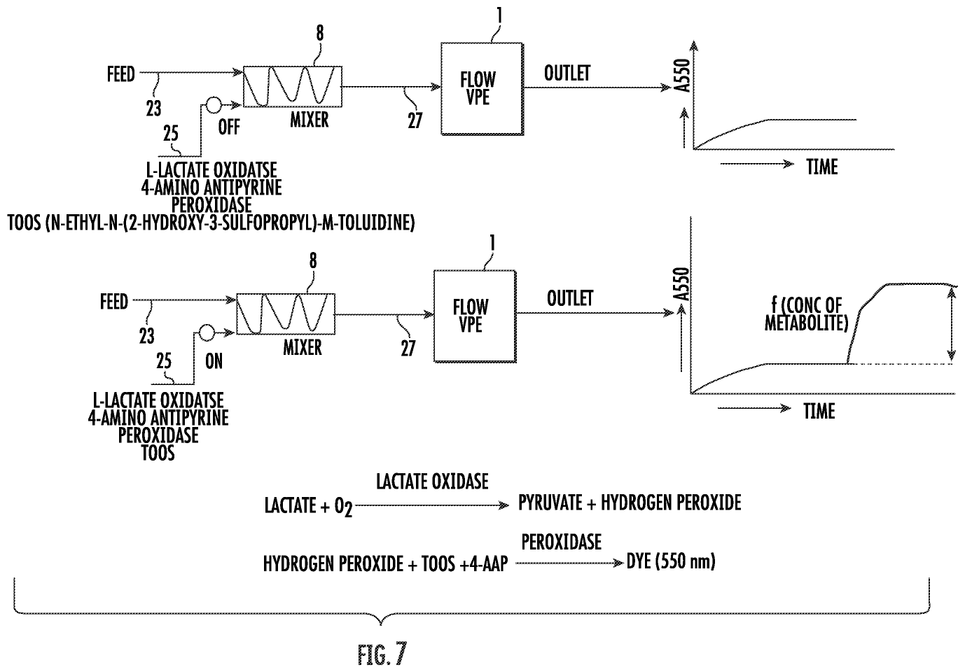

Absorbance of a first feed 23 containing lactate is read at 550 nm, as illustrated in FIG. 7. Lactate 23 is mixed via mixer 8 with reagents 25 including L-Lactate oxidase, 4-amino antipyrine (4-AAP), peroxidase, and N-ethyl-N-(2-hydroxy-3-sulfopropyl)-m-toluidine (TOOS). The lactate and oxygen may react with the lactate oxidase to produce pyruvate and hydrogen peroxide. The hydrogen peroxide, 4-AAP, and TOOS may react with the peroxidase to produce quinoneimine dye 27. The concentration of the quinoneimine dye may be estimated with an absorbance reading at 550 nm. The concentration of lactate in the first feed 23 is determined by the difference in absorbance signals between the initial reading and a reading of the product of the mixing step 27 at 550 nm.

Absorbance of a feed containing a species of interest may be measured at a wavelength. The feed may be treated via a filtering process, which may deplete the species of interest. The absorbance of the product of the treatment, such as filtrate of the filtering process, may be read at the wavelength. The concentration of the species of interest in the feed may be estimated based on the difference between the absorbance readings.

Absorbance of a feed containing a species of interest may be measured at a wavelength. The feed may be passed through a chromatography column, which may deplete the species of interest. The absorbance of the product of the treatment, such as flow through of the chromatographic process, may be read at the wavelength. The concentration of the species of interest in the feed may be estimated based on the difference between the absorbance readings.

Absorbance of a feed containing a species of interest may be measured at a wavelength. The feed may be treated such that the species of interest is substantially removed from the feed. The absorbance of the product of the treatment, such as filtrate of the filtering process, may be read at the wavelength. The concentration of the species of interest in the feed may be estimated based on the difference between the absorbance readings.

Absorbance of a feed containing a species of interest may be measured at a wavelength. At least one reagent may be mixed with the feed in a mixing chamber 8. The reagent may effect a reaction with the species of interest to generate a product discoverable at the same wavelength of the initial reading. The initial concentration of the species of interest in the feed may be estimated based on the difference between absorbance of the readings of the initial feed and the feed containing the product.

Absorbance of a feed containing a species of interest may be measured at a wavelength. At least one reagent may be added to the feed, wherein the reagent may effect a reaction with the species of interest to generate a product discoverable at the same wavelength of the initial reading. The initial concentration of the species of interest in the feed may be estimated based on the difference between absorbance of the readings of the initial feed and the feed containing the product.

Absorbance of a feed containing a species of interest may be measured at a wavelength. A reaction may be facilitated between at least one reagent and the species of interest to generate a product discoverable at the same wavelength of the initial reading. The initial concentration of the species of interest in the feed may be estimated based on the difference between absorbance of the readings of the initial feed and the feed containing the product.

Methods for estimating a concentration of a species of interest in a sample described herein include causing a change in the concentration measurable at a predetermined wavelength of an absorbance reading and estimating the change using a flow cell spectrometer.

1. A method of determining a sample concentration, comprising:
passing a first feed through a flow-through variable pathlength spectrophotometer, wherein the first feed comprises a sample and impurities;
reading a first absorbance value;
passing a second feed of the sample through the flow-through variable pathlength spectrophotometer, wherein the second feed comprises the impurities; and
reading a second absorbance value,
wherein the difference between the first absorbance value and the second absorbance value comprises a concentration of the sample.

2. The method of claim 1, further comprising determining a third absorbance value corresponding to a difference between the first absorbance value and the second absorbance value and using the third absorbance value to determine the concentration of the sample.

3. The method of claim 1, wherein the first feed comprises a cell culture fluid.

4. The method of claim 1, wherein reading the first and second absorbance values comprises measuring the respective absorbances at 280 nm.

5. The method of claim 1, further comprising passing the first feed through an affinity column prior to passing the first feed through the flow-through variable pathlength spectrophotometer.

6. The method of claim 5, wherein the affinity column comprises a lactate dehydrogenase (LDH) affinity column or a Protein A affinity column.

7. A method for determining a sample concentration, comprising:
passing a first fluid through a flow cell spectrometer, wherein the non-treated feed comprises a sample and impurities;
reading a first absorbance value;
passing the non-treated feed through an affinity column, wherein the resulting fluid comprises a treated feed;
passing the treated fluid through the flow cell spectrometer; and
reading a second absorbance value,
wherein the difference between the first absorbance value and the second absorbance value comprises a concentration of the sample.

8. The method of claim 7, further comprising determining a third absorbance value corresponding to a difference between the first absorbance value and the second absorbance value and using the third absorbance value to determine the concentration of the sample.

9. The method of claim 7, wherein the first feed comprises a cell culture fluid.

10. The method of claim 7, wherein reading the first and second absorbance values comprises measuring the respective absorbances at 280 nm.

11. The method of claim 7, wherein the affinity column comprises a lactate dehydrogenase (LDH) affinity column or a Protein A affinity column.

12. The method of claim 7, wherein the flow cell spectrometer comprises a flow through variable pathlength spectrophotometer

13. A method of determining a sample concentration, comprising:
passing a first fluid through a flow cell spectrometer, wherein the first fluid comprises a sample and impurities;
reading a first absorbance value;
mixing the first fluid with a second fluid, wherein mixing further comprises causing a reaction which produces a product;
passing the product through the flow cell spectrometer; and
reading a second absorbance value,
wherein the difference between the first absorbance value and the second absorbance value is proportional to a concentration of the sample.

14. The method of claim 13, wherein the first fluid comprises a non-treated feed fluid.

15. The method of claim 13, wherein the second fluid comprises a reagent

16. The method of claim 15, wherein the reagent comprises glucose oxidase, peroxidase, 4-aminopherazone, and phenol.

17. The method of claim 15, wherein the reagent comprises reduced nicotinamide adenine dinucleotide (NADH).

18. The method of claim 15, wherein the reagent comprises L-lactate oxidase, 4-amino antipyrine, peroxidase, and N-ethyl-N-(2-hydroxy-3-sulfopropyl)-3-methylaniline (TOOS).

18. The method of claim 13, further comprising determining a third absorbance value corresponding to a difference between the first absorbance value and the second absorbance value and using the third absorbance value to determine the concentration of the sample.

19. The method of claim 13, wherein reading the first and second absorbance values comprises measuring the absorbance at 340 nm, 505 nm, or 550 nm.

20. The method of claim 13, wherein the product comprises quinoneimine dye.
